(19) United States
(12) Patent Application Publication
(10) Pub. No.: US 2011/0125222 A1
(43) Pub. Date: May 26, 2011

(54) Transdermal Photonic Energy Transmission Devices and Methods

(75) Inventors: Rodney C. Perkins, Woodside, CA (US); Sunil Puria, Sunnyvale, CA (US)

(73) Assignee: SoundBeam LLC, Redwood City, CA (US)

(21) Appl. No.: 12/822,821

(22) Filed: Jun. 24, 2010

Related U.S. Application Data
(60) Provisional application No. 61/220,124, filed on Jun. 24, 2009.

(51) Int. Cl. A61N 1/00

(52) U.S. Cl. 607/61

(57) ABSTRACT

An output assembly comprises at least one light detector configured for placement under skin near a temporal bone so as to couple with a behind the ear unit coupled with the Pinna. The area of the at least one detector may comprise an area to couple with a light source. As the area of the detector under the skin can be large the at least one detector under the skin can couple efficiently with a light source. An input transducer assembly can be configured to transmit light energy to the output assembly with the multiplexed optical signal through the skin tissue. The multiplexed optical signal may comprise a pulse width modulated signal so as to decrease the effect of non-linearities of the light source and light detector and provide quality sound to the user.

### CROSS-REFERENCES TO RELATED APPLICATIONS

The present patent application is a non-provisional and claims priority to U.S. Pat. App. Ser. No. 61/220,124 filed 24 Jun. 2009, entitled “Transdermal Photonic Energy Transmission Device and Methods” (attorney docket no. 026166-003300US), the full disclosure of which is incorporated herein by reference.

### STATEMENT AS TO RIGHTS TO INVENTIONS MADE UNDER FEDERALLY SPONSORED RESEARCH AND DEVELOPMENT

NOT APPLICABLE

### BACKGROUND OF THE INVENTION

### Field of the Invention

The present invention relates to transdermal delivery of light energy to implantable devices generally, and more specifically to photonic light energy delivery to implantable devices that stimulate of the cochlea for hearing. Although specific reference is made to cochlear implants, embodiments of the present invention can be used in many applications wherein tissue is stimulated with energy, for example with stimulation of muscles, nerves and neural tissue, for example stimulation of the brain for the treatment of Parkinson's disease and heart disease.

The prior devices used to stimulate tissue can be somewhat invasive, in at least some instances. The implanted device may use power from an external source in at least some instances. At least some of the prior devices have placed a coil in tissue and transmitted power to the coil. With prior cochlear implants, energy can be transmitted through a pair of transmitter and receiver RF coils. In at least some instances the implantation of the coils can be somewhat invasive, and the coils can be somewhat larger than would be ideal. At least some of the implanted coils may have a smaller size to decrease invasiveness of the implanted coil. Consequently, alignment of the implanted coil with an external coil can be important in at least some instances. In at least some instances, a pair of magnets may be used to align the RF coils. One of the two magnets can be semi-permanently implanted in temporal bones in at least some instances. Body implanted magnets are contraindications for MRI machines, and thus the magnets can be surgically removed prior to imaging in at least some instances. Cochlear implants can be implanted in children as young as 18 months and implanted in adults, and at some point in a person's life, he or she will likely need an MRI in at least some instances. This use of surgically implanted magnets can result in a surgical procedure for magnet removal prior to a MRI and a second procedure for reimplantation in at least some instances.

Signal transmission for tissue stimulation with implanted coils can rely on implanted circuitry to convert signal received by the coil into electric impulses sent through an internal cable to electrodes of the cochlear implant. Such implanted circuitry can make the implanted device somewhat larger than would be ideal. For example, the implanted receiver may receive signal instructions from the speech processor with magnetic induction sent from the transmitter, in which the implanted receiver may be embedded in the skull behind the ear in at least some instances.

At least some of the prior cochlear implants may produce a perceived sound quality that is less than ideal in at least some instances. For example, in at least some instances speech recognition may be less than ideal. Also, in at least some instances, the prior devices may not provide sound localization cues that are present with natural hearing.

It would be helpful to stimulate tissue in a manner that overcomes at least some of the shortcomings of the prior devices.

### BRIEF SUMMARY OF THE INVENTION

The present invention relates to transdermal delivery of light energy to implantable devices generally, and more specifically to photonic light energy delivery to implantable devices that stimulate of the cochlea for hearing. Although specific reference is made to cochlear implants, embodiments of the present invention can be used in many applications wherein tissue is stimulated with energy, for example with stimulation of muscles, nerves and neural tissue, for example the treatment of Parkinson's disease and heart disease.

Embodiments of the present invention provide devices, systems and methods of stimulating tissue with light that overcome at least some of the problems associated with the prior devices. For example, an output assembly may comprise at least one light detector configured for placement under skin near a temporal bone, for example under skin near the pinna of the ear so as to couple with a behind the ear unit coupled with the Pinna. The area of the at least one detector may comprise an area to couple with a light source. As the area of the detector under the skin can be large the at least one detector under the skin can couple efficiently with a light source of a behind the ear (hereinafter “BTE”) unit. For example, the area of the at least one detector for coupling may comprise at least about 50 square mm, for example 100 square mm or more. Also, the output assembly may comprise substantially non-magnetic materials such that a person can undergo MRI imaging when the output assembly is implanted. An input transducer assembly can be configured to transmit light energy to the output assembly. For example, the input assembly can be configured to transmit the multiplexed optical signal through the skin tissue. The multiplexed optical signal may comprise a pulse width modulated signal so as to decrease the effect of non-linearities of the light source and light detector and provide quality sound to the user. The output assembly can be configured in many ways to stimulate tissue in response to the light transmitted through the skin. For example, the at least one photodetector can be coupled to an electrode array positioned in the cochlea. The at least one photodetector may be coupled to a light source to generate light energy within the body, and the light energy can be transmitted to a location within the body with at least one waveguide such as an optical fiber. The base band audio signal can be decomposed into a plurality of bandpass filtered channels and a high frequency pulse width modulated signal for each channel can be determined so as to preserve the amplitude and phase of the base band audio signal. With high frequencies stimulation above about 10 kHz, for example above about 20 kHz, the cochlea can low pass filter and demodulate the high frequency pulse width modulated signal into the base band audio sound signal with the amplitude and phase substantially maintained such that the patient can hear the sound with amplitude and phase of the base band audio signal. The at least one photodetector may be coupled to an actuator to vibrate tissue, for example such that the user hears sound corresponding to the location of the vibrated tissue.

In a first aspect, embodiments of the present invention provide a method of transmitting light energy to a device implanted in a user having a skin disposed over temporal bone. The light energy is transmitted through the skin disposed over the temporal bone to the implanted device.

In many embodiments, the skin comprises skin disposed over a mastoid process of the temporal bone.

In many embodiments, the light energy is generated with a light source. The light source may comprise at least one of a light emitting diode or a laser diode. In many embodiments, the light source comprises the laser diode.

In many embodiments, an output assembly is configured for placement at least partially behind a pinna of an ear of the user, and the output assembly comprises the laser diode.

In many embodiments, the light energy comprises infrared light energy.

In many embodiments, the light energy is received by at least one photodetector positioned under the skin. The implanted device may comprise an output assembly comprising the at least one photodetector. The at least one photodetector may comprise at least one of crystalline silicon, amorphous silicon, micromorphous silicon, black silicon, cadmium telluride, copper indium gallium selenide or indium gallium arsenide.

In many embodiments, the at least one photodetector is coupled to at least one of an implanted actuator, an implanted cochlear electrode or an implanted light source. For example, the at least one photodetector can be coupled to the implanted actuator, and the implanted actuator may comprise at least one of a coil, a coil and a magnet, a piezoelectric transducer, a balanced armature transducer or a magnetostrictive transducer. The implanted actuator can be positioned at least partially in the middle ear and configured to vibrate such that the user hears sound in response to the light energy transmitted through the skin.

In many embodiments, the at least one photodetector is coupled to the implanted cochlear electrode and wherein the implanted cochlear electrode comprises an array of implanted electrodes positioned at least partially within a cochlea of the user to stimulate cochlear tissue with the electrode array. The electrical current can be passed through electrodes of the implanted array such that the user hears sound in response to the light energy transmitted through the skin.

In many embodiments, the array of implanted electrodes comprises a plurality of pairs of electrodes, each pair of the plurality of electrodes can be coupled to a pair of opposing photo detectors to generate a biphasic current pulse between the pair of electrodes. Each pair of photodetectors and each corresponding pair of electrodes may correspond to a channel of the output assembly.

In many embodiments, the at least one photodetector is coupled to the implanted light source, and the implanted light source generates light energy in response to the light energy transmitted to the at least one photodetector.

In many embodiments, the at least one photodetector is coupled to the implanted light source, and the implanted light source emits light energy such that the user hears sound in response to the light energy transmitted through the skin. The implanted light source may comprise at least one of a light emitting diode or a laser diode, for example.

In many embodiments, the implanted light source comprises a plurality of light sources configured to emit a plurality of wavelengths of light.

In many embodiments, the implanted light source is coupled to at least one optical fiber extending at least partially into a cochlea of the user.

In many embodiments, the implanted light source generates multiplexed optical signal that comprises a plurality of channels, each channel of the plurality corresponding to at least one frequency of the sound. The plurality of channels may correspond to at least about sixteen channels, and said at least one frequency may corresponds to at least about sixteen frequencies, for example.

In many embodiments, the multiplexed optical signal comprises a time division multiplexed signal. A modulator can be coupled to the light source such that the modulator can adjust the light beam to emit light from an opening of the at least one optical fiber in response to the light energy transmitted to the at least one detector. The at least one optical waveguide may comprise a plurality of wavelength selective optical waveguides, and the modulator can adjust a wavelength of the light to direct the light substantially along each of the wavelength selective optical waveguides to an opening on a distal end of said wavelength selective optical waveguide. The light source may comprise a laser, and the opening may comprise a plurality of openings disposed along the at least one waveguide. The modulator can be configured to adjust a mode structure of the laser to transmit light substantially through one of the plurality of openings.

In many embodiments, the mode structure comprises a first mode structure and a second mode structure and the plurality of openings comprises a first opening and a second opening and wherein the at least one waveguide is configured to emit light substantially through the first opening in response to the first mode structure and through the second opening in response to the second mode structure.

In another aspect, embodiments provide a system to transmit an audio signal to a user having an ear and skin disposed over a temporal bone. An input assembly comprises at least one light source configured to emit a light energy, and the input assembly is configured for placement at least partially behind the ear of the user. An output assembly comprises at least one detector configured to receive the light energy to stimulate tissue, and the at least one detector is configured for placement at least partially under the skin disposed over the temporal bone to couple with the input assembly.

In many embodiments, the input assembly is configured to transmit a signal comprising a plurality of pulses of the light energy to stimulate the tissue, and the input assembly comprises circuitry configured to determine widths of the plurality of light pulses to transmit the signal to the at least one detector in response to an input signal.

In many embodiments, the skin comprises skin disposed at least partially over a mastoid process of the temporal bone.

In many embodiments, the at least one light source comprises at least one of a light emitting diode or a laser diode. The light source may comprise the laser diode, for example. The output assembly can be configured for placement at least partially behind a pinna of the ear of the user, and the output assembly may comprise the laser diode.

In many embodiments, the light energy comprises infrared light energy.

In many embodiments, the at least one photodetector comprises at least one of crystalline silicon, amorphous silicon, micromorphous silicon, black silicon, cadmium telluride, copper indium gallium selenide or indium gallium arsenide.

In many embodiments, the at least one photodetector is coupled to at least one of an implantable actuator of the output assembly, an implantable cochlear electrode of the output assembly, or an implantable light source of the output assembly to stimulate the tissue.

In many embodiments, the at least one photodetector is coupled to the implantable actuator and wherein the implantable actuator comprises at least one of a coil, a coil and a magnet, a piezoelectric transducer, a balanced armature transducer or a magnetostrictive transducer. The implantable actuator can be configured for placement at least partially in the middle ear and configured to vibrate such that the user hears sound in response to the light energy transmitted through the skin.

In many embodiments, the at least one photodetector is coupled to the implantable cochlear electrode, and the implantable cochlear electrode comprises an array of implantable electrodes configured for placement at least partially within a cochlea of the user to stimulate cochlear tissue with the electrode array.

In many embodiments, the output assembly comprises circuitry configured to pass electrical current through electrodes of the implantable array such that the user hears sound in response to the light energy transmitted through the skin. The array of implanted electrodes may comprise a plurality of pairs of electrodes, in which each pair of the plurality of electrodes is coupled to a pair of opposing photodetectors to generate a biphasic current pulse between said pair of electrodes. Each pair of photodetectors and each corresponding pair of electrodes may comprise a channel of the output assembly.

In many embodiments, the at least one photodetector is coupled to the implantable light source, and the implantable light source is configured to generate light energy in response to the light energy transmitted to the at least one photodetector. The output assembly comprises an optical array, and the implantable light source is configured to emit light energy through the optical array such that the user hears sound in response to the light energy transmitted through the skin. The implantable light source may comprise at least one of a light emitting diode or a laser diode, for example.

In many embodiments, the implantable light source comprises a plurality of light sources configured to emit a plurality of wavelengths of light.

In many embodiments, the implantable light source is coupled to at least one optical fiber configured to extend at least partially into a cochlea of the user.

In many embodiments, the implantable light source is configured to generate multiplexed optical signal comprising a plurality of channels, in which each channel of the plurality corresponds to at least one frequency of the sound. The plurality of channels may correspond to at least about sixteen channels, and said at least one frequency may correspond to at least about sixteen frequencies.

In many embodiments, the multiplexed optical signal comprise a time division multiplexed signal.

In many embodiments, a modulator is coupled to the light source and wherein the modulator is configured to adjust the light beam to emit light from an opening of the at least one optical fiber in response to the light energy transmitted to the at least one detector. The at least one optical waveguide may comprise a plurality of wavelength selective optical waveguides, and the modulator can be configured to adjust a wavelength of the light to direct the light substantially along each of the wavelength selective optical waveguides to an opening on a distal end of said wavelength selective optical waveguide. The light source may comprise a laser and the opening comprises a plurality of openings disposed along the at least one waveguide and wherein the modulator is configured to adjust a mode structure of the laser to transmit light substantially through one of the plurality of openings. The mode structure may comprise a first mode structure and a second mode structure, and the plurality of openings may comprise a first opening and a second opening. The at least one waveguide can be configured to emit light substantially through the first opening in response to the first mode structure and substantially through the second opening in response to the second mode structure.

In many embodiments, the implanted device comprises an output assembly comprising substantially non-magnetic materials configured for MRI imaging when implanted in the user.

In another aspect, embodiments provide a method of providing a device for a user. An incision is made in a skin of the user, and the skin disposed over a temporal bone. At least one photodetector is passed through the incision to position the at least one photo detector under skin disposed over the mastoid process of the temporal bone.

In many embodiments, at least one of an actuator, an electrode, or a light source are coupled to the at least one photodetector and implanted with the photodetector.

In another aspect embodiments provide a device to stimulate tissue. The device comprises means transmitting an optical signal and means for receiving the optical signal. The means for transmitting the optical signal may comprise one or more components of the input assembly, for example the complete input assembly, and the means for receiving the optical signal may comprise one or more components of the output assembly, for example the complete output assembly.

### BRIEF DESCRIPTION OF THE DRAWINGS

FIG. 1A shows an optically coupled implant system comprising a behind the ear unit and an output assembly, in accordance with embodiments of the present invention;

FIG. 1A1 shows an output transducer assembly configured to extend at least partially along tissue of the ear canal, such that at least a portion of the output transducer is covered with the tissue that extends at least partially along the ear canal EC;

FIG. 1A2 shows an optically coupled implant system comprising a behind the ear unit and output assembly comprising at least one waveguide configured to extend through a minimally invasive hole drilled in bone, in accordance with embodiments of the present invention;

FIG. 1A3 shows an optically coupled implant system comprising a behind the ear unit and output assembly comprising at least one waveguide configured to extend at least partially along tissue of the ear canal, such that at least a portion of the at least one waveguide is covered with the tissue that extends at least partially along the ear canal EC, in accordance with embodiments of the present invention;

FIG. 1A4 shows an optically coupled implant system comprising a behind the ear unit and output assembly comprising at least activator configured to vibrate the ear, in accordance with embodiments of the present invention;

FIG. 1B shows an input transducer assembly configured to emit a multiplexed optical signal, in accordance with embodiments of the present invention;

FIG. 1C shows an input transducer assembly configured to emit optical signal comprising one or more wavelengths of light multiplexed in accordance with embodiments of the present invention;

FIG. 1D optical pulses comprising separate wavelengths of light of a wavelength multiplexed optical signal as in FIG. 1C;

FIG. 1E shows an optical multiplexer configured to wavelength multiplex light from a plurality of light sources having separate wavelengths, as in FIG. 1C;

FIG. 1F shows an output transducer assembly comprising an optical demultiplexer comprising optical filters and an array of detectors, in accordance with embodiments;

FIG. 1F1 shows circuitry of a channel of the output transducer assembly of FIG. 1F so as to provide at least biphasic pulses in response to a first light pulse comprising a first wavelength and a second light pulse comprising a second wavelength, in accordance with embodiments;

FIG. 1F2 shows an electrode array comprising electrode pairs spaced apart for use with biphasic pulses, in accordance with embodiments;

FIG. 2A shows an input transducer assembly configured to emit a time multiplexed optical signal in accordance with embodiments of the present invention;

FIG. 2A1 optical pulses comprising a series of pulses of the time multiplexed optical signal as in FIG. 2A;

FIG. 2A2 shows a clock pulse of the series of optical pulses of the time multiplexed optical signal as in FIG. 2A;

FIG. 2B shows an output transducer assembly configured for use with an input transducer assembly as in FIG. 2A.

FIG. 3A shows an input transducer assembly configured to emit a time multiplexed optical signal in accordance with embodiments of the present invention;

FIG. 3A1 optical pulses comprising a series of pulses of the time multiplexed optical signal as in FIG. 3A;

FIG. 3A2 shows a clock pulse of the series of optical pulses of the time multiplexed optical signal as in FIG. 3A;

FIG. 3B shows an output transducer assembly configured for use with an input transducer assembly as in FIG. 3A;

FIG. 4 shows tri-phasic pulse width modulated pulses, in accordance with embodiments;

FIG. 5A shows signal to channel conversion with bandpass filtering and pulse width modulation so as to maintain substantially phase of the audio signal among the channels with high frequency stimulation of the cochlea, in accordance with embodiments; and

FIG. 5B shows pulses of a channel for high frequency stimulation of the cochlea so as to maintain phase of the audio signal as in FIG. 5A.

### DETAILED DESCRIPTION OF THE INVENTION

The present invention relates to transdermal delivery of light energy to implantable devices generally, and more specifically to photonic light energy delivery to implantable devices that stimulate of the cochlea for hearing. Although specific reference is made to cochlear implants, embodiments of the present invention can be used in many applications wherein tissue is stimulated with energy, for example with stimulation of muscles, nerves and neural tissue, for example the treatment of Parkinson's disease and heart disease.

As used herein light encompasses infrared light, visible light and ultraviolet light.

Embodiments can be configured to stimulate tissue in many ways based on the photon energy and wavelength of light. For example, the embodiments may include a optical signal comprising photons transmitted to at least one detector disposed under dermal tissue. The optical signal may comprise a multiplexed optical signal, and the multiplexed optical signal may comprise more than one wavelength of light so as to stimulate tissue based on the photonic properties of light. Examples of wavelength selective devices suitable for incorporate in accordance with embodiments include wavelength selective optical filters, gratings, etalons, waveguides and detectors.

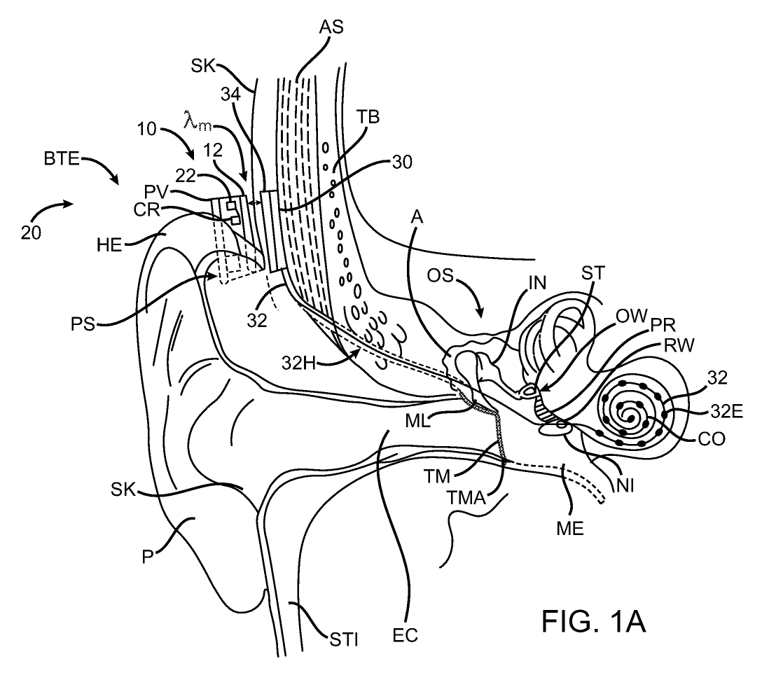

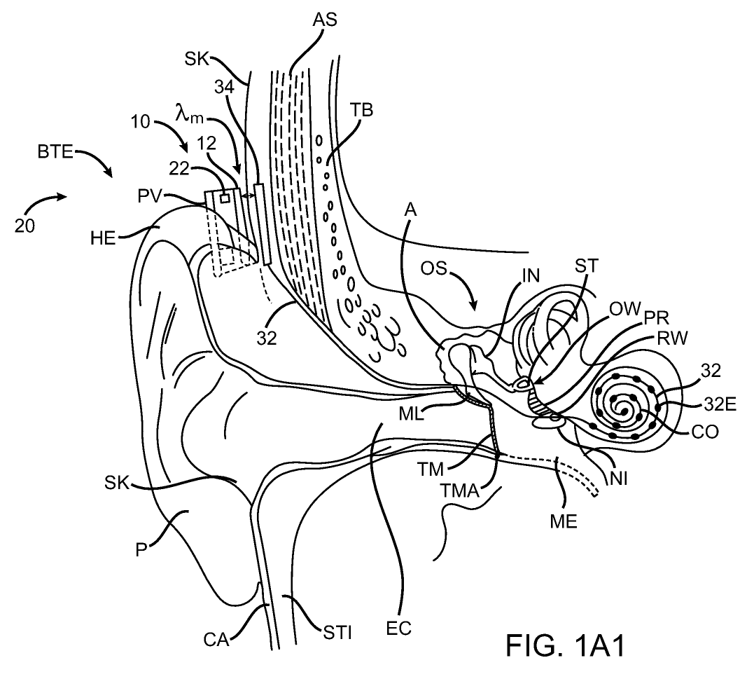

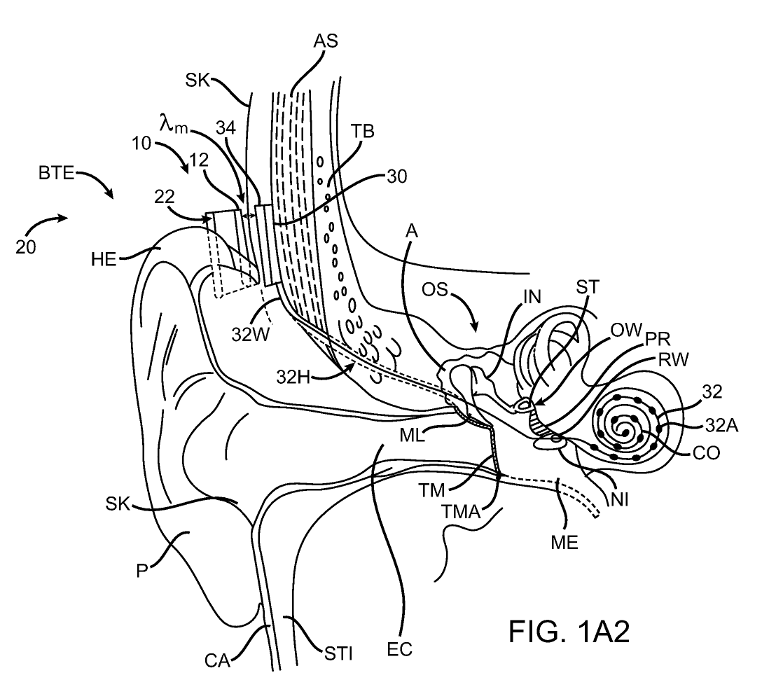

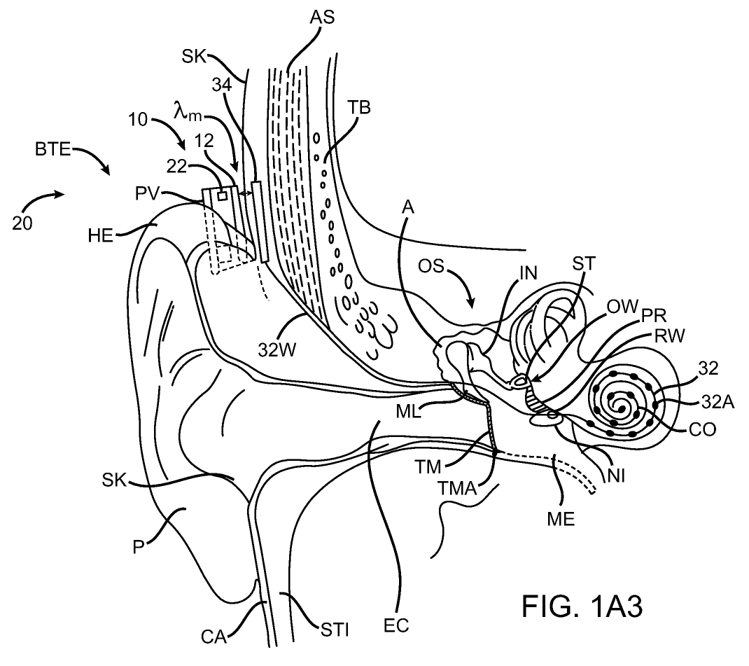

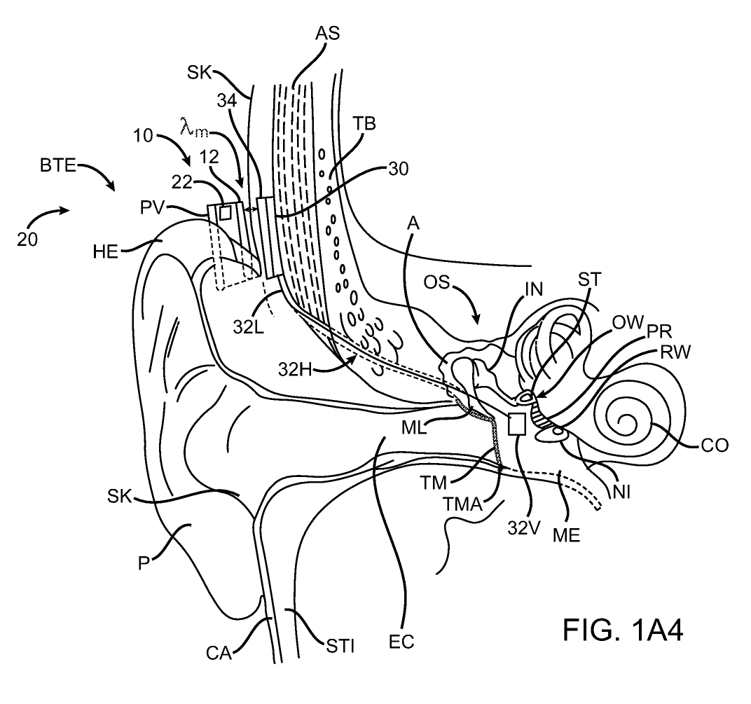

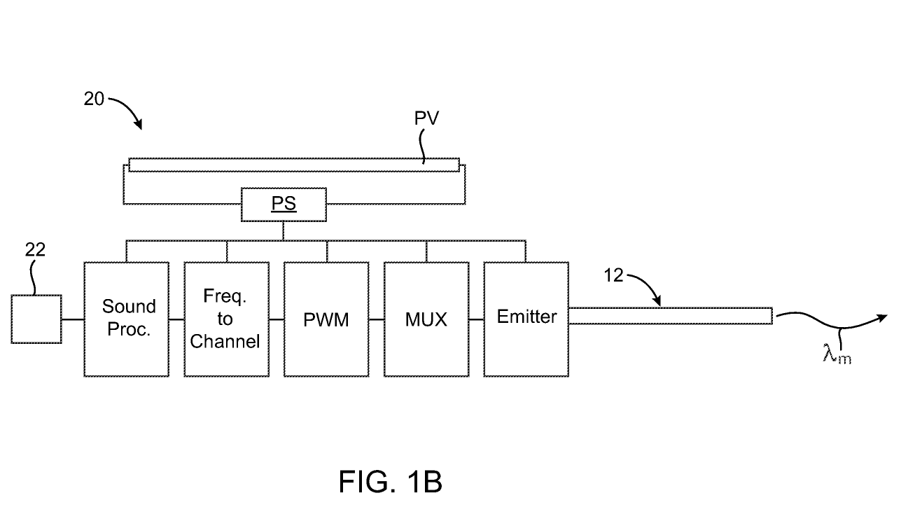

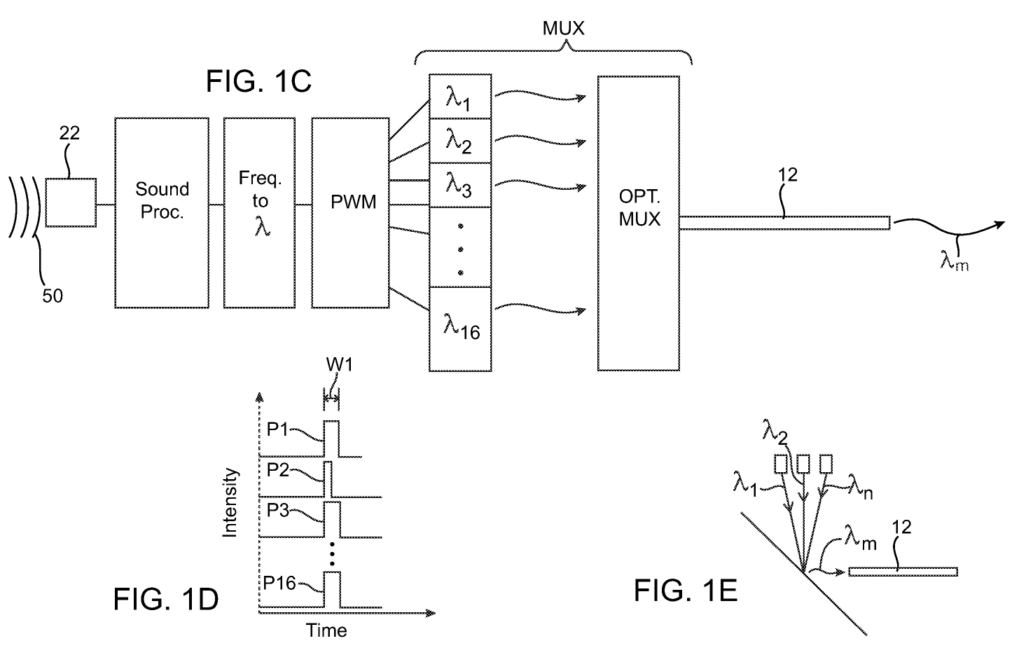

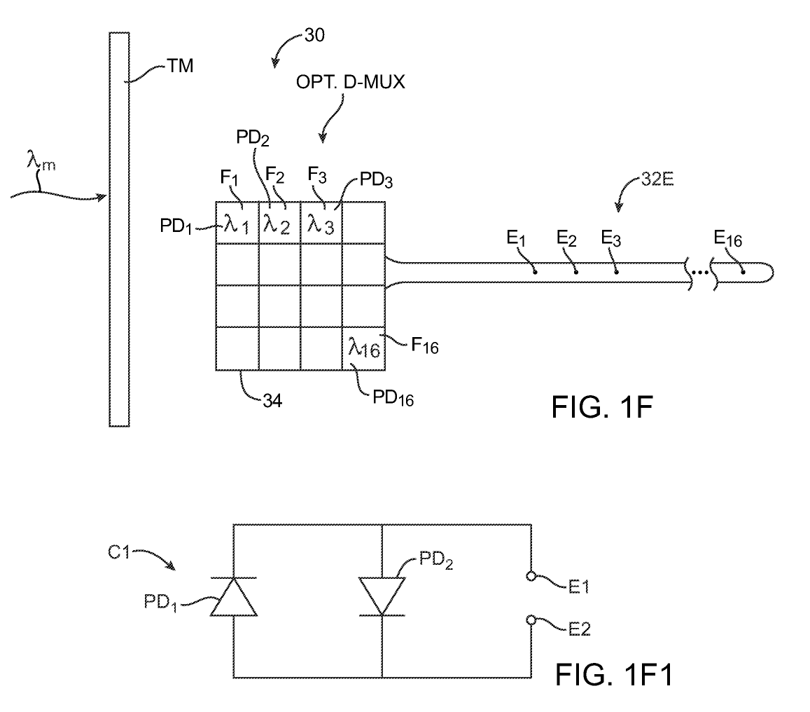

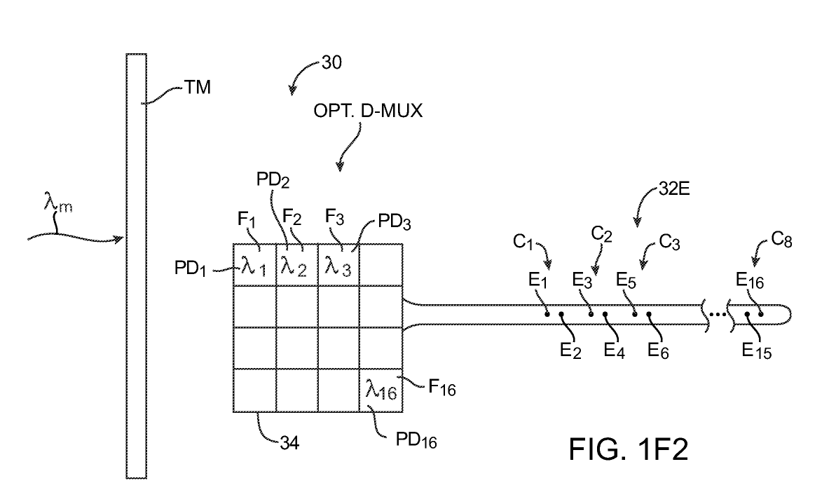

FIG. 1A shows an optically coupled cochlear implant system 10 comprising an input transducer assembly 20 and an output assembly 30. The input transducer assembly 20 may comprise behind the ear unit (hereinafter “BTE”). The BTE unit can be positioned behind a pinna P of the user, so as to decrease visibility of the BTE unit. The pinna P comprises a prominent rim, or helix H, and the BTE unit can be configured to hide at least partially behind helix H. The BTE unit can house electronics used to process and input signal. An input transducer, for example microphone 22, is coupled to the BTE unit and can transmit an audio signal to the BTE unit. The BTE can convert the input signal into a multiplexed optical signal λ_{M}. The BTE unit can be coupled to an optical transmission structure 12 to emit the multiplexed optical signal λ_{M}. The light transmission structure 12 can extends from the BTE into the ear canal EC. The light transmission structure 12 may support microphone 22. Microphone 22 can be positioned in many locations, for example within the ear canal or near the ear canal opening to detect sound localization cues. Alternatively, the microphone may be positioned on the ear canal. The input transducer may comprise a second microphone positioned on the BTE unit for noise cancellation. The sound input may comprise sound from a Bluetooth connection, and the BTE may comprise circuitry to couple with a cell phone, for example.

The output assembly 30 is configured for placement under the skin disposed over the temporal bone, and to extend through the middle ear to the inner ear of the user. The at least one detector 34 of the output assembly can be configured for placement over muscle tissue such as auricularis superior AS muscle tissue. The output assembly may extend at least partially through the muscle tissue. For example, the output assembly 30 can be configured to extend through a hole 32H formed in temporal bone TB to the middle ear ME. The hole 32H may extend to an attic A of the middle ear. The output assembly 30 comprises at least one detector 34 configured to receive the multiplexed optical signal λₘ. The output assembly comprises an electrode array 32 coupled to the at least one detector 34 so as to stimulate the cochlea in response to the multiplexed optical signal λ_{M}. The electrode array comprises a plurality of electrodes 32E, for example 16 pairs of electrodes. The output assembly 30 may comprise a demultiplexer coupled to the at least one detector to demultiplex the optical signal. The multiplexed optical signal may comprise, for example, a time multiplexed optical signal or a wavelength multiplexed optical signal. The demultiplexer comprises structures so as to demultiplex the optical signal and stimulate tissue of the cochlea. The demultiplexer can be configured to coupled pulses of the multiplexed optical signal with electrodes of the array such that pulses of the multiplexed optical signal correspond to electrodes of the array.

The output assembly 30 may comprise many known biocompatible and substantially non-magnetic materials, such that output assembly 30 is configured for use with MRI imaging when implanted in the patient. For example the electrode array 32 may comprise substantially non-magnetic conducting metal, such as at least one of Platinum, Titanium, Ni, or Nitinol. The electrode array may comprise a biocompatible substantially non-magnetic housing material, for example at least one of silicone elastomer, biocompatible plastic, or hydrogel.

The electrode array 32E and at least one photo detector 34 can be configured in many ways to stimulate the cochlea. For example, the electrodes can be coupled to the photo detector for monophasic pulses. The electrode array may comprise bi-phasic pulses with a first pulse corresponding to a first current in a first direction and a second pulse corresponding to a second pulse in a second direction.

The BTE unit may comprise circuitry (CR) that can be coupled to microphone 22. The circuitry may comprise a sound processor. The BTE unit may comprise an energy storage device PS configured to store electrical energy. The storage device may comprise many known storage devices such at least one of a battery, a rechargeable batter, a capacitor, a supercapacitor, or electrochemical double layer capacitor (EDLC). The BTE unit can be removed, for example for recharging or when the user sleeps. Feedback is substantially non-existent due to the electrical and non-acoustic stimulation of the cochlea, and the microphone 22 may be configured for placement in the ear canal EC.

The energy storage device PS may comprise a rechargeable energy storage device that can be recharged in many ways. For example, the energy storage device may be charged with a plug in connector coupled to a super capacitor for rapid charging. Alternatively, the energy storage device may be charged with an inductive coil or with a photodetector PV. The photodetector detector PV may be positioned on a proximal end of the ECM such that the photodetector is exposed to light entering the ear canal EC. The photodetector PV can be coupled to the energy storage device PS so as to charge the energy storage device PS. The photodetector may comprise many detectors, for example black silicone as described above. The rechargeable energy storage device can be provided merely for convenience, as the energy storage device PS may comprise batteries that the user can replace when the ECM is removed from ear canal.

The photodetector PV may comprise at least one photovoltaic material such as crystalline silicon, amorphous silicon, micromorphous silicon, black silicon, cadmium telluride, copper indium gallium selenide, and the like. In some embodiments, the photodetector PV may comprise black silicon, for example as described in U.S. Pat. Nos. 7,354,792 and 7,390,689 and available under from SiOnyx, Inc. of Beverly, Mass. The black silicon may comprise shallow junction photonics manufactured with semiconductor process that exploits atomic level alterations that occur in materials irradiated by high intensity lasers, such as a femto-second laser that exposes the target semiconductor to high intensity pulses as short as one billionth of a millionth of a second. Crystalline materials subject to these intense localized energy events may under go a transformative change, such that the atomic structure becomes instantaneously disordered and new compounds are “locked in” as the substrate re-crystallizes. When applied to silicon, the result can be a highly doped, optically opaque, shallow junction interface that is many times more sensitive to light than conventional semiconductor materials. Photovoltaic transducers for hearing devices are also described in detail in U.S. Patent Applications Nos. 61/073,271, entitled “Optical Electro-Mechanical Hearing Devices With Combined Power and Signal Architectures” (Attorney Docket No. 026166-001800US); and 61/073,281, entitled “Optical Electro-Mechanical Hearing Devices with Separate Power and Signal” (Attorney Docket No. 026166-001900US), the full disclosures of which have been previously incorporated herein by reference and may be suitable for combination in accordance with some embodiments as described herein.

FIG. 1A1 shows output assembly 30 configured to extend at least partially along tissue of the ear canal, such that at least a portion of the output transducer is covered with the tissue that extends at least partially along the ear canal EC

FIG. 1A2 shows an optically coupled implant system comprising a behind the ear unit and output assembly comprising at least one waveguide 32W configured to extend through hole drilled 32H in bone.

FIG. 1A3 shows an optically coupled implant system 10 comprising a behind the ear BTE unit and output assembly 30 comprising at least one waveguide 32W configured to extend at least partially along tissue of the ear canal EC, such that at least a portion of the at least one waveguide is covered with the tissue that extends at least partially along the ear canal EC.

FIG. 1A4 shows optically coupled implant system 10 comprising behind the ear unit BTE and output assembly 30 comprising at least activator 32V configured to vibrate the ear, in which the activator 32V is coupled to the at least one detector with line 32L comprising wires.

Line 32L can extend along hole 32H, as described above, to couple the at least one vibrator 32V with at least one photodetector 34.

FIG. 1B shows input transducer assembly 20 configured to emit a multiplexed optical signal. The components of the input transducer assembly 20 may be housed in BTE unit. The components may be power with power supply PS and photodetector such as a photovoltaic as described above. Microphone 22 is coupled to a sound processor. The sound processor may comprise one or more of many commercially available sound processors. The sound processor comprises a tangible medium to store instructions of a computer program embodied therein. The sound processor may comprise or be coupled to a multi-band frequency to channel converter. The frequency to channel converter can convert frequencies of the audio signal to filtered sound channels corresponding to locations of the cochlea for electrical stimulation such that the user perceives the sound of the audio signal. The circuitry of in input assembly may comprise pulse width modulation (hereinafter “PWM”) circuitry. The PWM circuitry can be configured to determine the width of each optical pulse corresponding to one of the electrodes of the array. The width of the optical pulse can be determined in response to the frequency of the sound corresponding to the electrode that is coupled to the optical pulse. A multiplexer MUX and an emitter are coupled to the PWM circuitry.

The emitter comprises at least one light source. The at least one light source emits pulses of light having a duration determined by the PWM circuitry. The width of the pulse refers to the duration of the pulse. With serial multiplexing, the at least one light source may comprise a single light source, and the timing of the pulses is determined by the multiplexer. With optical multiplexing, the at least one light source comprises a plurality of light sources, for example at least three light sources. The plurality of light sources can be configured to emit light pulses substantially simultaneously, or sequentially to decrease peak power consumption of the plurality of light sources.

The emitter is coupled to an optical transmission structure 12. The optical transmission structure may comprise an optical fiber, a plurality of optical fibers or a window, for example. The multiplexed light is transmitted from the optical transmission structure 12 toward tissue.

FIG. 1C shows an input transducer assembly 20 configured to emit a wavelength multiplexed optical signal. The sound processor can determine the frequencies of the audio signal. The multi-band filtered audio signal can be converted to channels of the electrode array and corresponding wavelengths with a frequency to wavelength converter (Freq. to λ). The width of each pulse for each wavelength is determined for a plurality of wavelengths, for example at least three wavelengths. Although sixteen wavelengths are shown, many more channels can be stimulated, for example up 32. The plurality of light sources comprises a first light source configured to emit first wavelengths λ1, a second light source configured to emit second wavelengths λ2, a third light source configured to emit third wavelengths λ3 and . . . a sixteenth light source configured to emit sixteenth wavelengths λ16. Light from each source is emitted to an optical multiplexer. The optical multiplexer may comprise many known methods of optical multiplexing. For example the optical multiplexer may comprise at least one of a grating, an etalon, a prism, an optical fiber, a waveguide, a nanostructure, or a plurality of optical fibers.

FIG. 1D optical pulses comprising separate wavelengths of light of a wavelength multiplexed optical signal as in FIG. 1C. A first pulse P1 comprises first wavelengths of light and a first width W1. A Second pulse P2 comprises second wavelengths of light and a second width. A third pulse P3 comprises third wavelengths of light and a third width. A fourth pulse P4 comprises fourth wavelengths of light and a fourth width. Additional pulses, for example a total of 16 or more, can be transmitted.

Each of the pulses comprise substantially separate pulses of light such that the pulses can be separated with the demultiplexer so as to correspond with one electrode of the array, or a pair of electrodes of the array. The wavelengths of each source may comprise wavelengths of a laser, in which the wavelengths of the laser correspond to the band width of the laser beam.

FIG. 1E shows an optical multiplexer configured to multiplex light from a plurality of light sources having separate wavelengths as in FIGS. 1C and 1D. Light from the sources can be emitted toward an optical structure grating, for example, and combined with optical transmission structure 12. The multiplexed signal can travel along optical transmission structure 12 toward the output assembly 30. The light for each channel of the multiplexed optical signal can be emitted serially from each source, so as to decrease peak power consumption of the light sources. For example the first light source can emit a first light pulse of the packet, followed by the second light source emitting the second light source of the packet until each of the light sources corresponding to one of the channels has emitted the corresponding pulse width modulated light signal of the packet. In many embodiments, each light source emits laser light when the other light sources of the optical multiplexer do not emit light. Thus serial use of the light sources can ensure that the power storage device can provide sufficient electrical energy to each of the light sources.

FIG. 1F shows an output transducer assembly 30 comprising an optical demultiplexer comprising optical filters and an array of detectors. The at least one detector 34 may comprise the array of detectors. The array of detectors comprises a first detector PD1, a second detector PD2, a third detector PD3 . . . and a sixteenth detector PD16. Additional or fewer detectors can be included in the array, for example 32 detectors. The optical multiplexer may comprise optical filters positioned in front of each detector to filter light transmitted to each detector. The optical multiplexer may comprise a first optical filter F1, a second optical filter F2, a third optical filter F3 . . . and a sixteenth optical filter F16. This configuration can separate the light into channels transmitted to each detector. For example, each filter can transmit wavelengths of light that are substantially separate from the wavelengths of light transmitted by the other filters. The array of electrodes comprises a first electrode E1, a second electrode E2, a third electrode E3 . . . and a sixteenth electrode E16. Each of the electrodes may comprise a pair of electrodes, for example 16 pairs of electrodes.

Each of the detectors is coupled to a corresponding electrode of the electrode array. First detector PD1 is coupled to first electrode E1 so as to comprise a first channel. Second detector PD2 is coupled to second electrode E2 so as to comprise a second channel. Third detector PD3 is coupled to third electrode E3 so as to comprise a third channel. The output assembly may comprise additional channels. For example, sixteenth detector D16 is coupled to sixteenth electrode E16 so as to comprise a sixteenth channel. Additional or fewer channels can be provided.

The perception of loudness due to electrical stimulation of the cochlea with electrodes 32E can depend on many factors including cochlear location, pulse width (duration) and pulse height (intensity). For pulses that are 50 us, for example, the current can be as high as 200 uA for a very loud sound. For a soft sound, only a 10 uA pulse can be sufficient. Increasing the width of the pulse can decrease the required amplitude of current.

Photodetectors can be configured to generate over 1 mA of current with a 4 mm² detector. Examples include a Si detector and an InGaAs detector. Sufficient current can be generated for multiple electrodes connected to corresponding detectors based on the detector area, the pulse width, and the efficiency detector and the intensity of the light beam on the detector. Based on the teaching described herein, a person of ordinary skill in the art can determine empirically the size of the photo detectors, the intensity and duration of the light pulses to provide a full spectrum of sound from soft to loud.

The electrode array 32E and at least one photo detector 34 can be configured in many ways to stimulate the cochlea with monophasic pulses or with bi-phasic pulses. For example, with 16 electrode pairs configured for bi-phasic pulses, the detector may comprise 16 pairs of detectors corresponding to 32 detectors. For example, each pair of electrodes can be coupled to two photodetectors, in which the two photodetectors are coupled to the electrodes with opposite polarity, such that a first light pulse to the first detector generates a first current between the electrodes in a first direction and a second light pulse to the second detector generates a second current between the two electrodes opposite the first current.

FIG. 1F1 shows circuitry of a channel of the output transducer assembly of FIG. 1F so as to provide at least biphasic pulses in response to a first light pulse comprising a first wavelength and a second light pulse comprising a second wavelength. The first channel C1 may comprise and/or correspond to a first pair of photodetectors comprising first photodetector PD1 and second photodetector PD1, and a first pair of electrodes comprising first electrode E1 and second electrode E2. The first photodetector PD1 can be coupled to electrode E1 and electrode E2 with a first polarity, and the second photodetector PD2 can be coupled to electrode E1 and electrode E2 with a second polarity opposite the first polarity so as to comprise a bipolar configuration of the first electrode and the second electrode. The first light pulse P1 of the first wavelength λ1 can generate current between electrode E1 and electrode E2 in a first direction, and the second light pulse P2 of the second wavelength λ2 can generate current between electrode E1 and electrode E2 in a second direction opposite the first direction. The width of the light pulses can be sized so as to balance charge between the electrodes and inhibit charge transfer, for example rectification. Additional channels can be provided with additional electrodes. For example, 8 channels can be provided with 8 pairs comprising 16 electrodes in a bipolar configuration, and the current can be generated in response to 16 wavelengths of light for example. Additional or fewer channels and corresponding electrodes and detectors may be provided, for example.

The photo detector array may comprise a first layer having a first array and a second layer having a second array. First wavelengths of can be absorbed by the first array, and the second wavelengths of light transmitted through the first array and absorbed by the second array, such that the combined array of the first array and second array can be decreased. Examples of detector materials having suitable properties are described in copending U.S. application Ser. No. 12/486,100 filed on Jun. 17, 2009, entitled, “Optical Electro-Mechanical Hearing Devices With Combined Power and Signal Architectures”, the full disclosure of which is incorporated herein by reference.

The stacked arrangement of detector arrays can be positioned on the output transducer assembly, and can provide greater surface area for each light output signal detected. For example, the combined surface area of the detectors may be greater than a cross-sectional area of the ear canal. The first detector array may be sensitive to light comprising wavelength of about 1 um, and the second detector array can be sensitive to light comprising wavelength of about 1.5 um. The first detector array may comprise a silicon (hereinafter “Si”) detector array configured to absorb substantially light having wavelengths from about 700 to about 1100 nm, and configured to transmit substantially light having wavelengths from about 1400 to about 1700 nm, for example from about 1500 to about 1600 nm. For example, the first detector array can be configured to absorb substantially light at 900 nm. The second detector array may comprise an Indium Gallium Arsenide detector (hereinafter “InGaAs”) configured to absorb light transmitted through the first detector and having wavelengths from about 1400 to about 1700 nm, for example from about 1500 to 1600 nm. The cross sectional area of the detector arrays can be about 4 mm squared, for example a 2 mm by 2 mm square for each detector array, such that the total detection area of 8 mm squared exceeds the cross sectional area of 4 mm squared of the detectors arrays in the middle ear. The detector arrays may comprise circular detection areas, for example a 2 mm diameter circular detector area. As the ear canal can be non-circular in cross-section, the detector arrays can be non-circular and rounded, for example elliptical with a size of 2 mm and 3 mm along the minor and major axes, respectively. The above detector arrays can be fabricated by many vendors, for example Hamamatsu of Japan (available on the world wide web at “hamamatsu.com”) and NEP corporation.

The light source and optical multiplexer of the input assembly can be configured in many ways to provide bandwidths suitable for use with two overlapping detector arrays. The light source and multiplexer can be combined with known wavelength multiplexing systems suited for incorporation in accordance with embodiments as described herein, such as components the EPIC integrated channelizer of the MIT Microphotonics Center and the photonics components available from Intel. The light source may comprise an integrated optical RF channelizer on silicon comprising an integrated photonics chip and laser light source. A first light laser source can be configured to emit light having wavelengths suitable for absorption with the first array, and the first light source can be coupled with a first modulator to modulate the first light beam so as to correspond to channels of the first array detector. A second light laser source can be configured to emit light having wavelengths suitable for transmission through the first array and absorption with the second array, and the second light source can be coupled with a second modulator to modulate the light beam so as to correspond to channels of the first array detector. The modulated light signals can be received by a multimode interferometeric splitter to demultiplex the transmitted light signal, for example. Transmission through the skin or through the skin and fat tissue can retain integrity of the transmitted light.

FIG. 1F2 shows an electrode array comprising electrode pairs spaced apart for use with biphasic pulses. Each of the electrodes of the pair may comprise a separation distance, and the distance from a first pair to a second pair can be greater than the separation distance between electrodes of the pair so as to provide sound frequency resolution comprising amplitude and phase. The pairs of electrodes may correspond to channels, for example a first channel C1, a second channel C2 and a third channel C3. The electrode pairs of each channel can be coupled to pairs photodetectors with opposite polarity as described herein so as to provide biphasic or triphasic pulses.

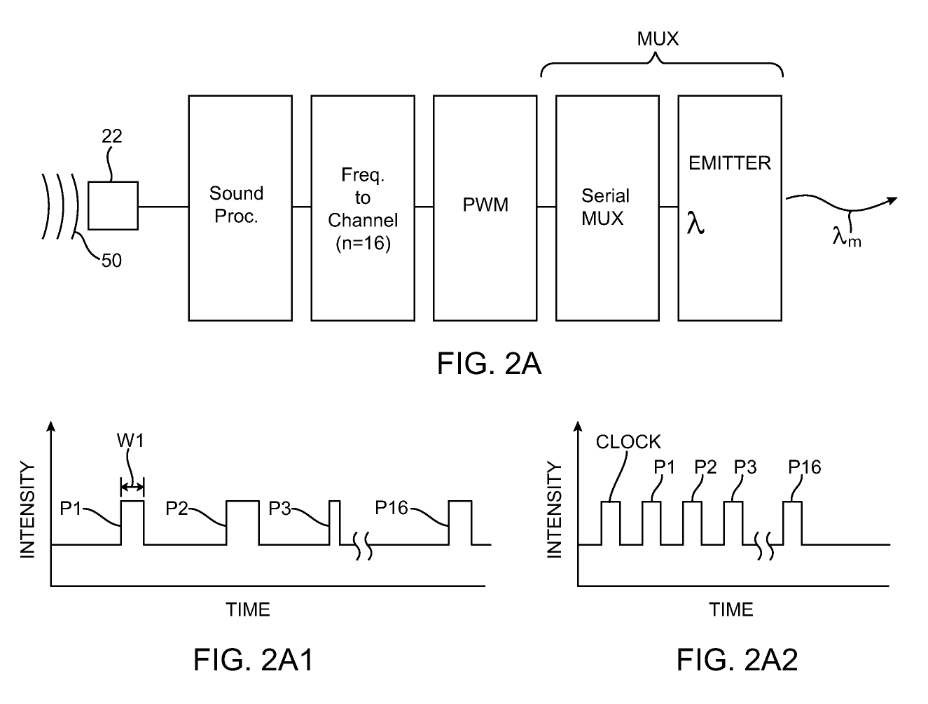

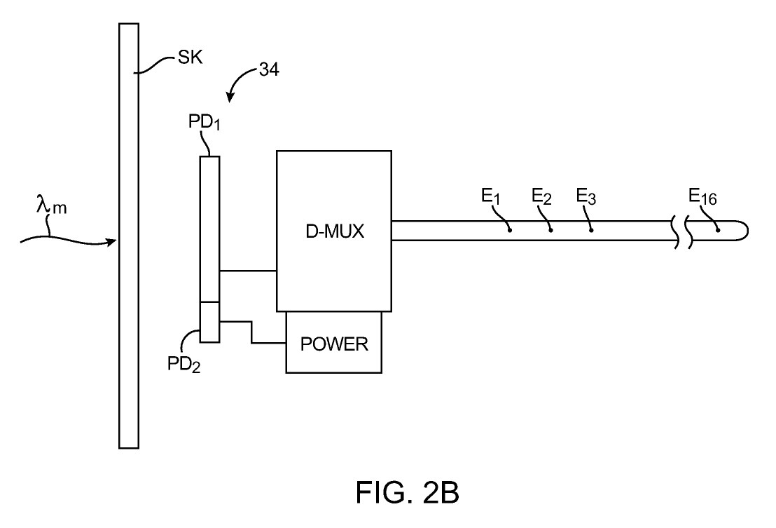

FIG. 2A shows an input transducer assembly configured to emit a time multiplexed optical signal. The multiplexed optical signal λM may comprise the time multiplexed optical signal, for example a serial multiplexed optical signal. An audio signal 50, for example a sound, is received by microphone 22. The audio signal comprises an input to the sound processor. The frequencies of the audio signal can be determined, for example with circuitry as described above.

The frequencies of the audio signal can be used to determine the amount of stimulation for each electrode of the array, in which each electrode corresponds to a channel. The width of each optical pulse can be determined with the PWM circuitry. The PWM circuitry is coupled to a serial multiplexer to multiplex the pulses for each electrode. The serial multiplexed pulses are emitted from an emitter comprising the at least one light source. The at least one light source may comprise a single light source, such as an infrared laser diode.

FIG. 2A1 optical pulses comprising a series of pulses of the time multiplexed optical signal as in FIG. 2A. The multiplexed serial pulses comprise a first pulse P1, a first pulse P1, a second pulse P2, a third pulse P3 . . . and a sixteenth pulse P16. Each pulse corresponds to one electrode of the array. An amount of electrical current is determined by a width of the pulse. First pulse P1 comprises a first width W1. Second pulse P2 comprises a second width. Third pulse P3 comprises a third width. Sixteenth pulse P16 comprises a sixteenth width. The multiplexer can be configured to emit packets of pulses, in which each packet comprises pulse information for each electrode of the array. For example, a packet may comprise sixteen pulses for the sixteen electrodes of the array. The serial multiplexer can be configured to emit the pulses of each packet so as to correspond with a predetermined timing and sequence of the pulses.

FIG. 2A2 shows a clock pulse of the series of optical pulses of the time multiplexed optical signal as in FIG. 2A. The clock pulse can synchronize the packet with the demultiplexer, such that the pulses are demultiplexed so as to correspond with the appropriate electrode. For example, pulse P1 may correspond with electrode E1. The clock pulse provide power to the demultiplexer circuitry.

FIG. 2B shows an output transducer assembly configured for use with an input transducer assembly as in FIG. 2A. The serial multiplexed optical signal is transmitted transdermally. The multiplexed optical signal is received by a photo detector PD1. Photodetector PD1 is coupled to demultiplexer circuitry D-MUX. The circuitry D-MUX may comprise a timer and switches such that the multiplexer sequentially couples each electrode to the detector in accordance with a predetermined sequence such that the detector is coupled to one of the electrodes when the pulse corresponding to the electrode is incident on detector PD1. For example, the pulse sequence may comprise a packet of pulses as described above. The first pulse of the packet may comprise a clock pulse to power the circuitry and to reset the timer. The timer can be coupled to the switches of the multiplexer such that a switch corresponding to one electrode is closed when the optical pulse corresponding to the electrode arrives at the detector. The timer and switches may comprise low power circuitry, for example CMOS circuitry, such that the timer and switches can be power with the clock pulse. This can be helpful when the audio signal is weak such that the timer and switching circuitry has sufficient power. Power storage circuitry such as capacitors and super capacitors can be coupled to the detector PD1 to store energy from the clock pulse with power circuitry (Power). The power circuitry can be switched with the switching circuitry such that the power storage capacitors are decoupled from the detector PD1 when the light pulses for the electrodes arrive at detector PD1.

The serial light source and detector components may comprise silicon photonics components of the MIT Microphotonics Center and the photonics components commercially available from Intel, as described above.

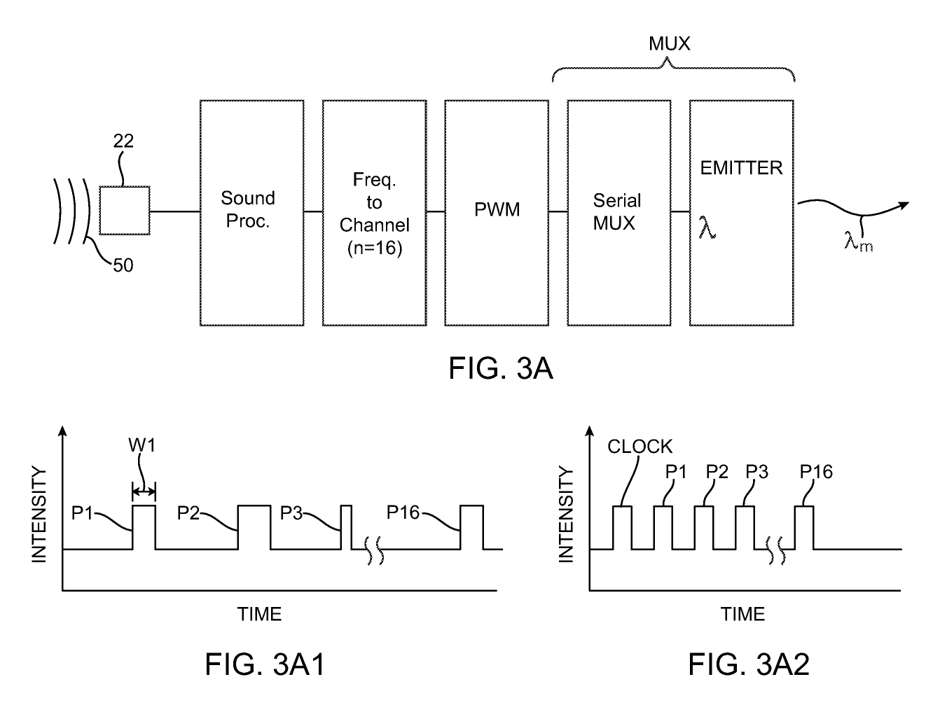

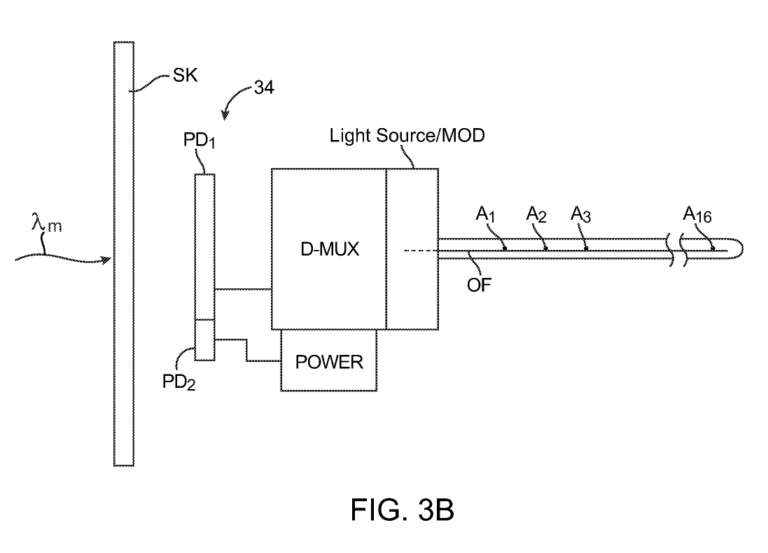

FIG. 3A shows an input transducer assembly configured to emit a time multiplexed optical signal. The multiplexed optical signal XM may comprise the time multiplexed optical signal, for example a serial multiplexed optical signal. An audio signal 50, for example a sound, is received by microphone 22. The audio signal comprises an input to the sound processor. The frequencies of the audio signal can be determined, for example with circuitry as described above. The frequencies of the audio signal can be used to determine the amount of stimulation for each electrode of the array, in which each electrode corresponds to a channel. The width of each optical pulse can be determined with the PWM circuitry. The PWM circuitry is coupled to a serial multiplexer to multiplex the pulses for each electrode. The serial multiplexed pulses are emitted from an emitter comprising the at least one light source. The at least one light source may comprise a single light source, such as an infrared laser diode.

FIG. 3A1 optical pulses comprising a series of pulses of the time multiplexed optical signal as in FIG. 3A. The multiplexed serial pulses comprise a first pulse P1, a first pulse P1, a second pulse P2, a third pulse P3 . . . and a sixteenth pulse P16. Each pulse corresponds to one electrode of the array. An amount of electrical current is determined by a width of the pulse. First pulse P1 comprises a first width W1. Second pulse P2 comprises a second width. Third pulse P3 comprises a third width. Sixteenth pulse P16 comprises a sixteenth width. The multiplexer can be configured to emit packets of pulses, in which each packet comprises pulse information for each electrode of the array. For example, a packet may comprise sixteen pulses for the sixteen apertures of the array. The serial multiplexer can be configured to emit the pulses of each packet so as to correspond with a predetermined timing and sequence of the pulses.

FIG. 3A2 shows a clock pulse of the series of optical pulses of the time multiplexed optical signal as in FIG. 3A. The clock pulse can synchronize the packet with the demultiplexer, such that the pulses are demultiplexed so as to correspond with the appropriate electrode. For example, pulse P1 may correspond with electrode E1. The clock pulse provide power to the demultiplexer circuitry.

FIG. 3B shows an output transducer assembly configured for use with an input transducer assembly as in FIG. 3A. The serial multiplexed optical signal is transmitted through one or more of the skin or the fascia. The multiplexed optical signal is received by a photo detector PD1. Photodetector PD1 is coupled to demultiplexer circuitry D-MUX. The demultiplexer circuitry D-MUX can be coupled to a light source and optical modulator so as to emit a modulated beam along at least one optical fiber OF in response to the multiplexed light signal. The modulator can be configured to modulate the light beam of the source such that light is emitted substantially from one of the openings so as to stimulate neural tissue of the cochlea in proximity to the opening.

The at least one optical fiber OF may comprise a plurality of openings configured to emit light in response to the light transmitted along the at least one fiber OF. For example, the at least one fiber OF may comprise a first opening A1, a second opening A2, a third opening A3, and a sixteenth opening A16. The at least one fiber OF may comprise additional or fewer openings as may be beneficial.

The circuitry D-MUX can be configured in many ways to demultiplex the optical signal. The circuitry D-MUX may comprise a timer and switches such that the multiplexer sequentially couples each electrode to the detector in accordance with a predetermined sequence such that the detector is coupled to one of the electrodes when the pulse corresponding to the electrode is incident on detector PD1. For example, the pulse sequence may comprise a packet of pulses as described above. The first pulse of the packet may comprise a clock pulse to power the circuitry and to reset the timer. The timer can be coupled to the switches of the multiplexer such that a switch corresponding to one electrode is closed when the optical pulse corresponding to the electrode arrives at the detector. The timer and switches may comprise low power circuitry, for example CMOS circuitry, such that the timer and switches can be power with the clock pulse. This can be helpful when the audio signal is weak such that the timer and switching circuitry has sufficient power. Power storage circuitry such as capacitors and super capacitors can be coupled to the detector PD1 to store energy from the clock pulse with power circuitry (Power). The power circuitry can be switched with the switching circuitry such that the power storage capacitors are decoupled from the detector PD1 when the light pulses for the openings arrive at detector PD1.

The output assembly can be configured in many ways to generate light in response to the multiplexed light signal. For example, the circuitry may comprise a light source and a modulator. The light source and modulator can be configured to emit light a series of light pulses corresponding to the openings of the optical fiber. The modulator can be configured to adjust the mode structure of the light source so as to emit light substantially from one of the apertures. The at least one optical fiber OF may comprise a plurality of waveguides, in which each waveguide is configured to transmit selectively light having a very narrow range of wavelengths. The modulator can adjust the wavelength of the light slightly such that light is transmitted along the waveguide corresponding to one of the openings so as to stimulate tissue with the narrow wavelengths of light corresponding to the channel of the opening.

A light source can be positioned in the middle ear and coupled to the optical array positioned at least partially within the cochlea. The light source in the middle ear can emit light in response to the time division multiplexed optical signal. A modulator can be positioned in the middle ear and coupled to the light source. The modulator can adjust the light beam to emit light from an opening of the at least one optical fiber in response to the time division multiplexed optical signal. The at least one optical waveguide may comprise a plurality of wavelength selective optical waveguides, for example photonic waveguides. The modulator can adjust a wavelength of the light to direct the light substantially along one of the wavelength selective optical waveguides to an opening on a distal end of the waveguide.

The light source may comprise a laser, and the opening may comprise a plurality of openings disposed along the at least one waveguide. The modulator can be configured to adjust a mode structure of the laser to transmit light substantially through one of the plurality of openings to stimulate the tissue, for example nerve tissue of the cochlea.

The serial light source and detector components may comprise silicon photonics components of the MIT Microphotonics Center and the photonics components commercially available from Intel, as described above.

In some embodiments, the power circuitry can be coupled to a separate detector PD2. The separate power and signal can be used to power the timing and switching circuitry.

The switching circuitry may comprise optical switches, for example an liquid crystal material, to switch the light signal transmitted to the optical fibers.

The light source may comprise a plurality of light sources coupled to a plurality of optical fibers extending into the cochlea, for example 16 light sources coupled to 16 optical fibers, as described in U.S. App. No. 61/218,377, filed Jun. 18, 2009, entitled “Optically Coupled Cochlear Implant Systems and Methods”, the full disclosure of which is incorporation by reference and may be suitable in accordance with some embodiments described herein.

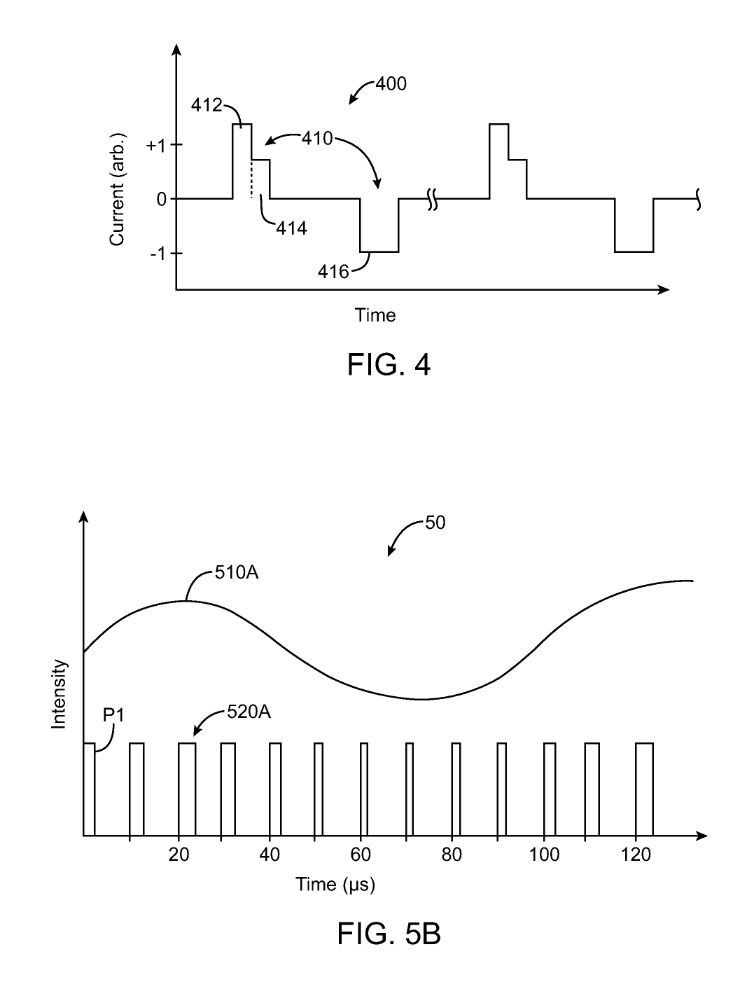

FIG. 4 shows tri-phasic pulse width modulated current pulses 400 corresponding to a channel of the electrode array. Each of the channels may comprise a pair of electrodes and the current pulses can be transmitted between the pair of electrodes corresponding to the channel. The tri-phasic current pulses 400 may comprise a first positive current pulse 412, a second positive current pulse 414 and a third negative current pulse 416. The first positive current pulse 412 and the second positive current pulse 410 comprise a positive amplitude to inject current may comprise a first amplitude and a second amplitude. Alternatively or in combination, the first positive current pulse 412 and the second positive current pulse 414 may comprise a substantially similar amplitude substantially different widths. The third negative current pulse comprises a negative polarity to balance the current and decrease degradation of the electrodes and tissue near the electrodes. The first positive current pulse and the second positive current pulse may transfer a first amount of charge and a second amount of charge with the current, respectively, and the third negative current pulse may transfer a third amount of current so as to balance the charge of the first pulses and decrease charge build up of the electrodes. As the area under a current pulse corresponds to the delivered charge of the current pulse, the cumulative area of the first positive current pulse and second positive current pulse may correspond substantially to the area of the cumulative area of the third negative pulse.

The photodetectors and filters can be coupled to the electrodes so as to pass at least biphasic current between the electrodes. Each of the plurality of channels may correspond to a pair of electrodes, and a first current can travel between said pair of electrodes in response to a first width modulated light pulse corresponding to positive current pulse 412 and a second current corresponding to negative pulse 416 may travel between said pair of electrodes in response to a second width modulated light pulse, as described above with reference to FIG. 2C and FIG. 2C1, for example. The first width modulated light pulse corresponding to positive pulse 412 may comprise a first wavelength of light and the second width modulated light pulse corresponding to negative pulse 416 may comprise a second wavelength of light. The second current pulse 414 may correspond to a second light pulse having the first wavelength, for example. The first current is opposite the second current. The first current has a first amount corresponding to a first width of the first light pulse and the second current has a second amount corresponding to a second width the second light pulse. The width of the first light pulse corresponds substantially to the width of the second light pulse so as to inhibit rectification and balance charge transfer between the first electrode and the second electrode.

The first light pulse may comprise a first wavelength of light coupled to a first detector, in which the second detector is coupled to said pair of electrodes. The second light pulse may comprise a second wavelength of light coupled to a second detector, in which said second detector is coupled to said pair of electrodes. The first detector is coupled to said pair of electrodes opposite the second detector. Each channel may correspond to a pair of electrodes and a first detector coupled to the pair of electrodes opposite a second detector, for example at least about 8 channels corresponding to 8 pairs of electrodes coupled 16 detectors.

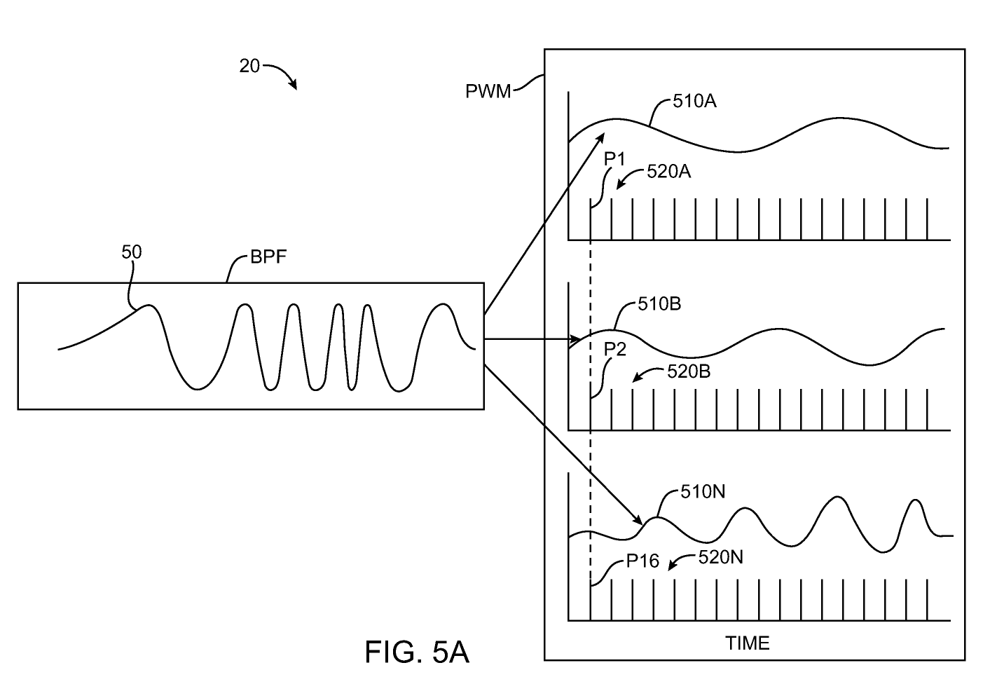

FIG. 5A shows signal to channel conversion with bandpass filtering and pulse width modulation so as to maintain substantially phase of the audio signal among the channels with high frequency stimulation of the cochlea. The input sound SO comprises a baseband sound signal. Work in relation to embodiments as described herein indicates that the cochlea can respond to high frequency electrical stimulation so as to low pass filter the high frequency stimulation and demodulate the high frequency signal into the baseband signal, such that the person can perceive sound having the amplitude and phase of the base band signal preserved based on the high frequency electrical stimulation, for example electrical stimulation having frequencies above the range of hearing of the patient. For example, with stimulation of high frequencies above about 10 kHz, for example above about 20 kHz, the cochlea can low pass filter the sound such that the patient hears the sound with magnitude and phase of the audio signal. When these high frequencies comprise magnitude and phase encoded information of the baseband audio signal, the user can hear the audio signal with the corresponding phase when the cochlea low pass filters and demodulates the high frequency signal into the base band audio signal. The high frequency signal above about 10 kHz, for example above 20 kHz, such as 40 kHz or 100 kHz or more, may comprise a pulse width modulated signal with amplitude and phase encoding with high frequencies, and the stimulation of the cochlea with the width modulated pulses at these high frequencies can result in demodulation of the high frequency pulse width modulated signal back into an audio band signal corresponding to the frequencies of the bandpass filtered channel. This demodulation of the high frequency amplitude and phase encoded signal can maintain both the amplitude and phase of the audio signal perceived by the user.

The audio signal 50 corresponding to a sound may comprise many frequencies and can be input into a bandpass filter BPF. The bandpass filter BPF may provide as output a first channel comprising first band pass audio signal 510A comprising a first range of frequencies, a second channel comprising a second band pass audio signal 510B comprising a second range of frequencies, and an Nth channel comprising an Nth band pass audio signal 510A comprising an Nth range of frequencies. Each of the signals may comprise a substantially similar phase such that the phase of the BPF output is substantially maintained.

The audio signal of each channel is converted to a pulse with modulated signal such that the phase of the original audio signal 50 is maintained among the channels. First bandpass audio signal 510A corresponds to a first series 520A of width modulated pulses. Second bandpass audio signal 510B corresponds to a second series 520B of width modulated pulses. Nth bandpass audio signal 510N corresponds to an Nth series 520N of width modulated pulses.

Each of the pulses may be determine so as to correspond to a substantially synchronous time base, such that each of the phase and amplitude of the original signal is maintained. For example, each of the pulses may be output to a corresponding light source to drive a corresponding photodetector, as described above. The Nth channel may comprise an eight channel, a sixteenth channel, a thirty second channel or a sixty fourth channel for example.

FIG. 5B shows a first series of width modulate pulses 520A of the first bandpass audio signal 510A of the first channel for high frequency stimulation of the cochlea so as to maintain phase of the audio signal as in FIG. 5A. The pulses may correspond to a synchronous a time base of 10 us between the leading edge of each pulse. The width of the pulses can vary based on the amplitude of the first bandpass filtered audio signal 510A. The corresponding frequency of the pulses is about 100 kHz and the pulses are demodulated by the cochlea with cochlear low pass filtering such that the user perceives sound with phase of the sound maintained and such that the user can perceive sound localization cues.

The bandpass filtered signals of the other channels can be processed similarly with cochlear low pass filtering of the high frequency signal such that the user perceives sound with the magnitude and phase of the sound maintained for each of the channels and such that the user can perceive sound localization cues from the combined channels.

While the pulse width modulated light pulses can be generated in many ways, the speech processor may comprise digital bandpass filters to output the bandpass filtered signal as an array for each channel, and the pulse width modulation circuitry can determine a width of each pulse of each channel based on the output, for example. As the output of the pulse width modulation circuitry can be digital and stored in the random access memory of the processor, the pulses to the light source can be delivered so as to maintain substantially the amplitude and phase of the output pulse modulation signal. For example, the timing and/or phase of the pulses of the signal can be maintained to within about 100 us for a 10 kHz pulse width modulation signal, and within about 10 us for a 100 kHz. Although the serial output among the channels may be used as described above and the timing and/or phase of each of the pulses of the channels may be shifted slightly relative to each other, the timing and/or phase of the corresponding pulses among the channels is substantially maintained with the serial output. For example, the corresponding light pulses of the serial output among the channels can be maintained to within about 100 us, for example within about 50 us, within about 20 us, or within about 10 us. The number of channels may comprise 2 channels, 4 channels, 8 channels, 16 channels, 32 channels or more for example. The frequency of the light pulses of each channel can be above at least about 10 kHz, for example 20 kHz, 40 kHz, 80 kHz, for example. The channels may be combined having the frequency of the light pulses of each channel as described above, such that the frequency of the width modulated pulses of the multiplexed optical signal transmitted across the eardrum may comprise, 40 kHz, 160 kHz, 640 kHz, 1280 kHz, or more, for example. Based on the teachings described herein, a person of ordinary skill in the art can determine the number of channels and the timing and/or phase of the pulses to maintain the phase of the audio signal when the cochlea is stimulated, for example so as to provide sound localization cues and so as to inhibit distortion.

Human Skin Transmission Experiments

Based on the teachings described herein, a person of ordinary skill in the art can conduct experiments to determine empirically transmission through the skin and placement depth of the detector, and also transmitter and detector sizes so as to transmit signals as described herein. For example, the inventors have performed experiments with the tympanic membrane and IR light having a wavelength of about 1500 nm and shown that at least about 50% of the light energy can be transmitted through the tympanic membrane when the detector is positioned within about 3 mm of the tympanic membrane, and this percentage can increase to at least about 70% when the detector is within about 1 mm of the tympanic membrane.

With transdermal illumination of the subdermal detector, the amount of light received by the detector can depend on the size of the detector relative to the size of the tissue illuminated and the depth of the detector from the dermal layer. As the molecular constituents of derma and skin are similar, the experiments with the tympanic membrane indicate that transdermal coupling efficiency can be at least about 25% between the source over the skin and detector under the skin, for example at least about 50%. The detector may comprise an area greater than the area of tissue illuminated, and the detector can be positioned under the skin with no more than about 2 mm of tissue between the detector and the dermal layer comprising skin. A person of ordinary skill in the art can determine empirically the position of the light source on the skin and the position of the detector under the skin so as to determine the light transmission, detector size and distance from the dermal layer comprising skin so as to provide multiplexed coupling through the skin as described herein.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims and the full scope of the equivalents thereof.

1. A method of transmitting light energy to a device implanted in a user having a skin disposed over temporal bone, the method comprising:
transmitting the light energy through the skin disposed over the temporal bone to the implanted device.

2. The method of claim 1 wherein the skin comprises skin disposed over a mastoid process of the temporal bone.

3. The method of claim 1 further comprising generating the light energy with a light source.

4. The method of claim 3 wherein the light source comprises at least one of a light emitting diode or a laser diode.

5. The method of claim 4 wherein the light source comprises the laser diode.

6. The method of claim 5 further comprising an output assembly configured for placement at least partially behind a pinna of an ear of the user and wherein the output assembly comprises the laser diode.

7. The method of claim 1 wherein the light energy comprises infrared light energy.

8. The method of claim 1 wherein the light energy is received by at least one photodetector positioned under the skin.

9. The method of claim 8 wherein the implanted device comprises an output assembly comprising the at least one photodetector.

10. The method of claim 8 wherein the at least one photodetector comprises at least one of crystalline silicon, amorphous silicon, micromorphous silicon, black silicon, cadmium telluride, copper indium gallium selenide or indium gallium arsenide.

11.-29. (canceled)

30. A system to transmit an audio signal to a user having an ear and skin disposed over a temporal bone, the system comprising:
an input assembly comprising at least one light source configured to emit a light energy, the input assembly configured for placement at least partially behind the ear of the user;
an output assembly comprising at least one detector configured to receive the light energy to stimulate tissue, the at least one detector configured for placement at least partially under the skin disposed over the temporal bone to couple with the input assembly.

31. The system of claim 30 wherein the input assembly is configured to transmit a signal comprising a plurality of pulses of the light energy to stimulate the tissue and wherein the input assembly comprises circuitry configured to determine widths of the plurality of light pulses to transmit the signal to the at least one detector in response to an input signal.

32. The system of claim 30 wherein the skin comprises skin disposed at least partially over a mastoid process of the temporal bone.

33. The system of claim 30 wherein the at least one light source comprises at least one of a light emitting diode or a laser diode.

34. The system of claim 33 wherein the light source comprises the laser diode.

35. The system of claim 34 wherein the output assembly is configured for placement at least partially behind a pinna of the ear of the user and wherein the output assembly comprises the laser diode.

36. The system of claim 30 wherein the light energy comprises infrared light energy.

37. The system of claim 30 wherein the at least one photodetector comprises at least one of crystalline silicon, amorphous silicon, micromorphous silicon, black silicon, cadmium telluride, copper indium gallium selenide or indium gallium arsenide.

38. The system of claim 30 wherein the at least one photodetector is coupled to at least one of an implantable actuator of the output assembly, an implantable cochlear electrode of the output assembly, or an implantable light source of the output assembly to stimulate the tissue.

39. The system of claim 38 wherein the at least one photodetector is coupled to the implantable actuator and wherein the implantable actuator comprises at least one of a coil, a coil and a magnet, a piezoelectric transducer, a balanced armature transducer or a magnetostrictive transducer.

40.-57. (canceled)

58. A method of providing a device for a user, the method comprising:
making an incision in a skin of the user, the skin disposed over a temporal bone of the user; and
passing at least one photodetector through the incision to position the at least one photo detector under skin disposed over the mastoid process of the temporal bone.

59. (canceled)

60. A device to stimulate tissue, the device comprising:
means transmitting an optical signal; and
means for receiving the optical signal.
